(19) United States
(12) Patent Application Publication
(10) Pub. No.: US 2013/0087141 A1
(43) Pub. Date: Apr. 11, 2013

(54) BACK SUPPORT BELT

(71) Applicant: Michael Williams, Logan, UT (US)

(72) Inventor: Michael Williams, Logan, UT (US)

(21) Appl. No.: 13/267,347

(22) Filed: Oct. 6, 2011

(51) Int. Cl. A61F 5/00

(52) U.S. Cl. 128/100.1

(57) ABSTRACT

A lumbar support belt is disclosed which circumscribes the waist and lower back of a wearer. The lumbar support belt comprises laterally extending panels, which are themselves circumscribed by laterally extending and interlocking wings which increase tension on the waist. The lumbar support belt prevents injuries to a wearer's lumbar, thoracic, and sacral areas of the back, as well to posterior cartilage and epithelial tissue.

### BACKGROUND OF THE INVENTION

1. Field of the Invention

This invention relates to back supports, and more particularly relates to an improved back support belt comprising internal and external belts.

2. Description of the Related Art

Traditional back belts or braces known in the art comprise elastomeric, vinyl or leather belts wrapped around the waist or lumbar posterior region of user's back. These belts provide support to the lumbar and thoracic areas of a user's body. Most of these support belts comprises laterally extending panels, which are interlock using Velcro, buckles, ties, or snaps.

Because traditional back supports are made to be durable, the durable materials forming them are not comfortable for wearers. Traditional supports are difficult to adjust, and to do not stretch or give as the user moves from one position to another. Traditional belt often do not comprise buckling or fastening mechanisms of sufficient strength to endure the pressures exerted on the belt as a user exerts himself

It is therefore desirable that a durable support belt be provided, which can be adjusted for comfort and for tension, and which comprises multiple fastening mechanisms for increased strength.

### SUMMARY OF THE INVENTION

From the foregoing discussion, it should be apparent that a need exists for an improved back support belt. Beneficially, such a belt would overcome many of the difficulties with prior art by providing a means for adjusting the comfort of the belt without comprising durability.

The present invention has been developed in response to the present state of the art, and in particular, in response to the problems and needs in the art that have not yet been fully solved by currently available information management systems. Accordingly, the present invention has been developed to provide a support device for the lumbar region of a user's back, the device comprising: a back panel adapted to generally circumscribe the posterior lumbosacral region of a user; a left panel attached to the back panel adapted to support the left side of the user's torso; a right panel attached to the back panel adapted to support the right side of a user's torso; fastening means for fastening the left panel to the right panel, the fastening means comprising one of Velcro, a buckle, and a tie; a left wing attached to the back panel, the left wing overlaying the left panel, the left wing adapted to support the left side of the user's torso; and a right wing attached to the back panel, the right wing overlaying the right panel, the right wing adapted to support the right side of the user's torso.

The support device further comprises a fastening means for fastening the left wing to the right wing, the fastening means comprising one of Velcro, a buckle, and a tie.

The support device may further comprise a tab affixed to one of the left wing and the right wing. The support device may further comprise a left wing defining a recess for receiving a distal end of the right wing.

The left wing may define a recess for receiving a distal end of the right wing.

A support brace for the lumbar region of a user's back is also disclosed, the brace comprising: a back panel adapted to generally circumscribe the posterior lumbosacral region of a user; a left panel attached to the back panel adapted to support the left side of the user's torso; a right panel attached to the back panel adapted to support the right side of a user's torso; fastening means for fastening the left panel to the right panel, the fastening means comprising one of Velcro, a buckle, and a tie.

The brace further comprises an exterior belt, affixed to the back panel, for circumscribing the waist of a user, the belt comprising: a left wing overlaying the left panel, the left wing adapted to support the left side of the user's torso; a right wing overlaying the right panel, the right wing adapted to support the right side of the user's torso; and fastening means for fastening the left wing to the right wing, the fastening means comprising one of Velcro, a buckle, and a tie.

The left wing may be necked is at its distal end.

The right wing may be necked is at its distal end.

These features and advantages of the present invention will become more fully apparent from the following description and appended claims, or may be learned by the practice of the invention as set forth hereinafter.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a side elevational perspective view of an improved back support belt in the open position in accordance with the present invention;

FIG. 2 is a side elevational perspective view of an improved back support belt in the fastened position accordance with the present invention;

FIG. 3A is a side elevational perspective view of the obverse side of an improved back support belt in accordance with the present invention;

FIG. 3B is a side elevational perspective view of the inverse side of an improved back support belt in accordance with the present invention; and

FIG. 4 is a side elevational environmental perspective view of an improved back support belt in accordance with the present invention.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

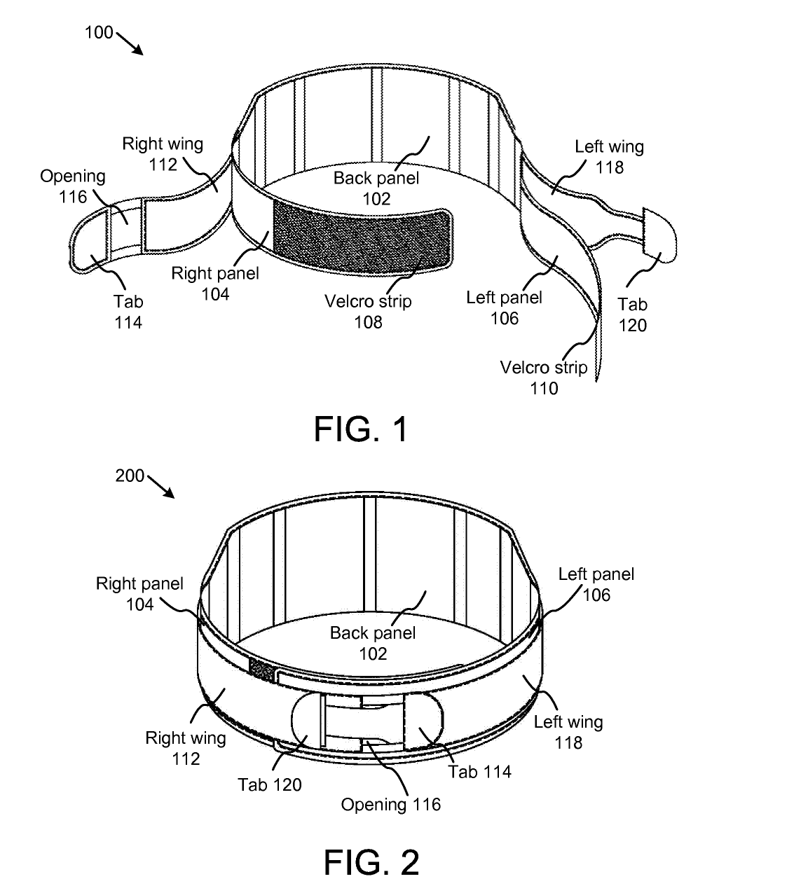

FIG. 1 is a side elevational perspective view of an improved back support belt 100 in the open position in accordance with the present invention. The back support belt 100 comprises a back panel 102, a right panel 104, a left panel 106, a Velcro strip 108, a Velcro strip 110, a right wing 112, a tab 114, an opening 116, a left wing 118, and a tab 120.

The back panel 102, the right panel 104, the left panel 106, the right wing 112, the tab 120, the tab 114, and the left wing 118 each comprise one of vinyl, nylon, elastomeric striping, leather, or any other flexible, durable material known to those of skill in the art. In the preferred embodiment, the back brace comprises flexible material or strips, with an inner surface and outer surface.

The right panel 104 and the left panel 106 may comprise separate panels of material. In other embodiments, the right panel 104 and left panel 106 comprise extensions of the back panel 102. The left panel 104 and the right panel 106 may be generally rectangular, square, triangular, or irregularly shaped.

The back panel 102, the left panel 104, and the right panel 106, are wrapped around the waist of a user or wearer. Together the panels 102-106 support the posterior lumbosacral region of the user's back when the user is lifting or carrying heavy objects.

The tightness, or snugness, of the belt 100 can be controlled with the fastening means attached to the distal ends of the left panel 104 and the right panel 106, such as the Velcro strips 108-110.

In various embodiments of the present invention, the belt 100 is circumscribed by an exterior belt comprising a left wing 118 and a right wing 112. In other embodiments, the belt 100 comprises the left wing 118 and the right wing 112. The wings 112, 118 may be permanently affixed or detachably attached to the back panel 102.

The wings 112, 118 comprise flexible leather, vinyl, polymer/elastomeric strips or panels which overlay the panels 104, 106. Like the panels 104, 106, the wings 112, 118 comprise fastening means, which may include one or more of Velcro, buckles, snaps, ties, or other fasteners known to those of skill in the art.

The opening 116 comprises a cut out portion, hole, annularity, or recess through which the distal, or detached, end of the right wing can be inserted before the right wing and left wing are fastened together using means known to those of skill in the art, including buckles, Velcro, snaps, ties, and the like.

In various embodiments, one or more of the left wing 118 and the right wing 112 may comprise a bore, hole, annularity, recess, or opening 116 through which the tip of the opposing right wing 112 or left wing 118 may be inserted. This recess may be square, rectangular or circular in shape, and may be one centimeter to 100 centimeters in area and/or 1 centimeter to 100 centimeters in diameter. In various embodiments, the left wing 118 and/or the right wing 112 comprise a plurality of openings 116.

FIG. 2 is a side elevational perspective view of an improved back support belt in the fastened position accordance with the present invention. The back support belt 200 comprises a back panel 102, a right panel 104, a left panel 106, a right wing 112, a tab 114, an opening 116, a left wing 118, and a tab 120.

The back panel 102, right panel 104, left panel 106, right wing 112, tab 114, opening 116, left wing 118, and tab 120 are all substantially described above in relation to FIG. 1.

FIG. 2 shows the belt 100, 200 in a closed configuration, in which the wings 112, 118 and the panels 104, 106 are fastened. In the shown embodiment, each is fastened using Velcro.

In various embodiments, the tab 120 is wider than the opening 116.

The back panel 102 may comprise one or more metal, alloy or wooden support bar(s) 202 traversing the back panel 102 in a direction orthogonal to the length of the belt 100 to keep the belt 200 from folding over on itself.

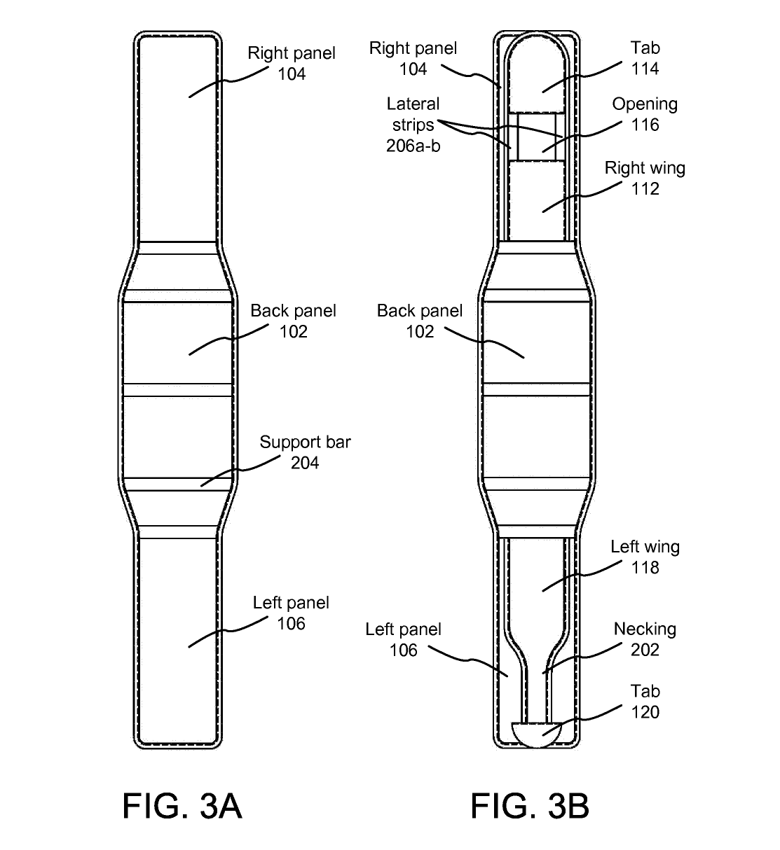

FIG. 3A is a side elevational perspective view of the obverse side of an improved back support belt in accordance with the present invention. The back support belt 300 comprises a back panel 102, a right panel 104, a left panel 106, and a support bar 204.

The back panel 102, right panel 104, left panel 106, right wing 112, tab 114, opening 116, left wing 118, and tab 120 are all substantially described above in relation to FIGS. 1-2.

FIG. 3A shows the obverse, or outside of the belt 100.

FIG. 3B is a side elevational perspective view of the inverse side of an improved back support belt in accordance with the present invention. The back support belt 350 comprises a back panel 102, a right panel 104, a left panel 106, a right wing 112, a tab 114, an opening 116, a left wing 118, a tab 120, necking 202, and lateral strips 206a-b.

The back panel 102, right panel 104, left panel 106, right wing 112, tab 114, opening 116, left wing 118, and tab 120 are all substantially described above in relation to FIGS. 1-3A.

FIG. 3B shows the inverse, or outside of the belt 100.

The lateral strips 206a-b define the opening 116, and are permanently affixed between the right wing 112 and the tab 114. The lateral strips are fabricated from the same material as the panels 104, 106.

In various embodiments, the left wing 118 is necked, or narrows, at it approaches the tab 120 at the distal end of the left wing 118.

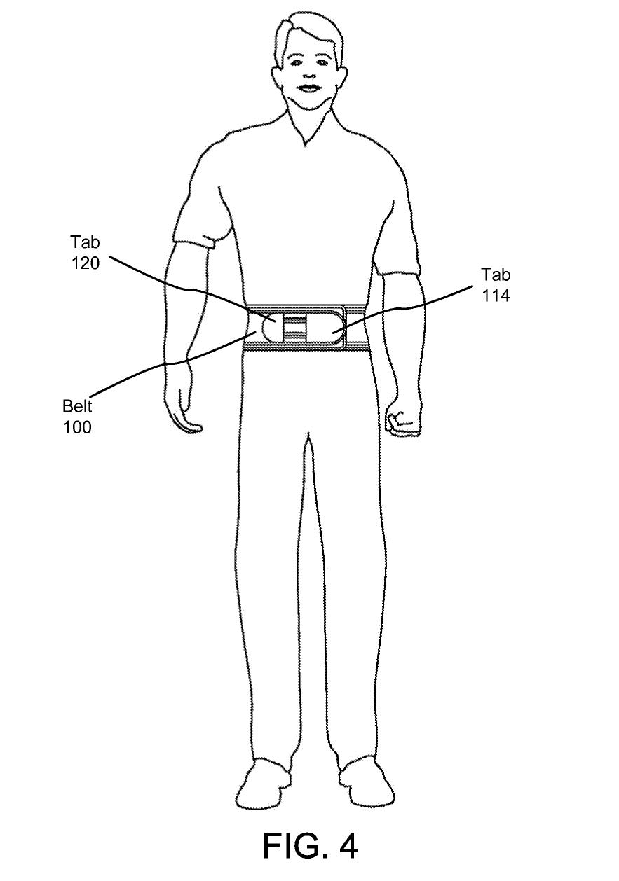

FIG. 4 is a side elevational environmental perspective view of an improved back support belt in accordance with the present invention. The back support belt 100 comprises a tab 120 and a tab 114.

The left and right panels 102, 104 may comprise a soft and supportive under layer that provides comfort and support to the use. The panels 102, 104 and the wings 112, 118 cross over in front of the user wearing the brace and Velcro on the front and/or each side.

The belt 100 allows the patient to pull and attach both straps at the same time thereby adjusting the comfort and support to the desired level. The design allows for more support than conventional back braces. Using this design allows the brace to be made in a two layer more supportive design as well as a single layer more athletic friendly design.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A support device for the lumbar region of a user's back, the device comprising:
a back panel adapted to generally circumscribe the posterior lumbosacral region of a user;
a left panel attached to the back panel adapted to support the left side of the user's torso;
a right panel attached to the back panel adapted to support the right side of a user's torso;
fastening means for fastening the left panel to the right panel, the fastening means comprising one of Velcro, a buckle, and a tie;
a left wing attached to the back panel, the left wing overlaying the left panel, the left wing adapted to support the left side of the user's torso;
a right wing attached to the back panel, the right wing overlaying the right panel, the right wing adapted to support the right side of the user's torso; and
fastening means for fastening the left wing to the right wing, the fastening means comprising one of Velcro, a buckle, and a tie.

2. The support device of claim 1, further comprising a tab affixed to one of the left wing and the right wing.

3. The support device of claim 1, wherein the left wing defines a recess for receiving a distal end of the right wing.

4. The support device of claim 1, wherein the left wing defines a recess for receiving a distal end of the right wing.

5. A support brace for the lumbar region of a user's back, the brace comprising:
a back panel adapted to generally circumscribe the posterior lumbosacral region of a user;
a left panel attached to the back panel adapted to support the left side of the user's torso;
a right panel attached to the back panel adapted to support the right side of a user's torso;
fastening means for fastening the left panel to the right panel, the fastening means comprising one of Velcro, a buckle, and a tie;
an exterior belt, affixed to the back panel, for circumscribing the waist of a user, the belt comprising:
a left wing overlaying the left panel, the left wing adapted to support the left side of the user's torso;
a right wing overlaying the right panel, the right wing adapted to support the right side of the user's torso; and
fastening means for fastening the left wing to the right wing, the fastening means comprising one of Velcro, a buckle, and a tie.

6. The brace of claim 5, wherein the left wing is at its distal end.

7. The brace of claim 5, wherein the right wing is at its distal end.
